(19) United States
(12) Patent Application Publication
(10) Pub. No.: US 2024/0122934 A1
(43) Pub. Date: Apr. 18, 2024

(54) COMPOSITIONS AND METHODS OF TREATMENT FOR INHIBITING CAPILLARY TUBE REGRESSION

(71) Applicant: University of South Florida, Tampa, FL (US)

(72) Inventors: George E. Davis, Tampa, FL (US); Gretchen Margaret Koller, Genevieve, MO (US)

(21) Appl. No.: 17/768,344

(22) PCT Filed: Oct. 16, 2020
(86) PCT No.: PCT/US2020/056077

§ 371 Date: Apr. 12, 2022

Related U.S. Application Data
(60) Provisional application No. 62/915,932, filed on Oct. 16, 2019.

(51) Int. Cl. A61K 31/522; A61K 31/27; A61K 31/352; A61K 31/4015; A61K 31/422; A61K 31/437; A61K 31/4418; A61K 31/4439; A61K 31/506; C07K 16/24

(52) U.S. Cl. CPC A61K 31/522; A61K 31/27; A61K 31/352; A61K 31/4015; A61K 31/422; A61K 31/437; A61K 31/4418; A61K 31/4439; A61K 31/506; C07K 16/247

(57) ABSTRACT

Compositions and methods of treatment for inhibiting capillary tube regression and/or lymphatic tube regression are described, as well as factors and signaling pathways that control the regression of capillary tube networks. Capillary tube regression and/or lymphatic tube regression may be implicated in ischemia, infarction, hypertension, diabetes, malignant cancer, neurodegenerative disease, wound repair response, atherosclerosis, pro-inflammatory disease, pro-thrombotic disease, viral infection (e.g., influenza or SARS-CoV-2), bacterial infection.

### CROSS-REFERENCE TO RELATED APPLICATION

This application claims priority to U.S. Provisional Patent Application No. 62/915,932, filed on Oct. 16, 2019. The content of this application is incorporated by reference in its entirety.

### FEDERALLY SPONSORED RESEARCH OR DEVELOPMENT

This invention was made with government support under Grant No. HL128584, awarded by the National Institutes of Health. The government has certain rights in the invention.

### BACKGROUND

Considerable progress has been made in recent years understanding the underlying molecular basis for blood vessel formation and maturation. How endothelial cells form tubes and sprout and how they attract mural cells to create mature blood vessels has been investigated and reviewed extensively. In contrast, very little work has focused on the converse of vessel formation, which is the molecular basis for vessel regression. A molecular understanding of vessel regression may lead to new therapeutic opportunities in many clinical contexts where capillary regression has been implicated as a key pathogenic feature. Capillary loss or rarefaction is observed in major human diseases including ischemia and infarction, hypertension, diabetes, malignant cancer, neurodegenerative diseases and wound repair responses. In addition, capillary regression plays a role in physiologic contexts including hyaloid vessel regression, and the menstrual cycle. Capillary regression mechanisms that occurred secondary to degradation of the extracellular matrix environment in which capillaries are embedded through the action of matrix metalloproteinases (MMPs) (e.g. MMP-1 and MMP-10) and MMP activators including serine proteinases such as plasmin have been previously investigated.

Pro-inflammatory mediators are known to have a profound influence on the vasculature and cause increased vascular permeability, leukocyte adhesion and transmigration, increased pro-coagulant activities, and increased platelet adhesion and aggregation. Key mediators include interleukin-1 (IL-1), tumor necrosis factor (TNF), and thrombin as well as many other cytokines and small molecule mediators that regulate these processes in health and disease states. Many recent studies indicate the therapeutic benefits in humans of neutralizing antibodies or pharmacologic antagonists directed to interleukin-1 beta (IL-1β), tumor necrosis factor alpha (TNFα), and thrombin in key disease states including atherosclerosis, pro-inflammatory diseases (e.g. arthritis, Crohn's disease, psoriasis), pro-thrombotic diseases (e.g. deep venous thrombosis, pulmonary embolism), and malignant cancer.

### SUMMARY

In some embodiments, presented herein are methods of treating a disease in a patient in need thereof, comprising administering to the patient a therapeutically effective amount of an inhibitor of capillary tube regression.

In some embodiments, presented herein are methods for treating a disease, the method comprising administering to a patient identified or diagnosed as having capillary tube regression a therapeutically effective amount of an inhibitor of capillary tube regression.

In some embodiments, presented herein are methods of treating a disease in a patient, the method comprising: a) confirming the presence of capillary tube regression in the patient; and b) administering to the patient a therapeutically effective amount of an inhibitor of capillary tube regression.

In some embodiments, presented herein are methods of treating a disease in a patient in need thereof, the method comprising administering to the patient a therapeutically effective amount of an inhibitor of lymphatic tube regression.

In some embodiments, presented herein are methods of treating a disease, the method comprising administering to a patient identified or diagnosed as having capillary tube regression a therapeutically effective amount of an inhibitor of lymphatic tube regression.

In some embodiments, presented herein are methods of treating a disease in a patient, the method comprising: c) confirming the presence of lymphatic tube regression in the patient; and d) administering to the patient a therapeutically effective amount of an inhibitor of lymphatic tube regression.

In some embodiments, presented herein are methods of the above, wherein the disease comprises ischemia, infarction, hypertension, diabetes, malignant cancer, neurodegenerative disease, wound repair response, atherosclerosis, pro-inflammatory disease, pro-thrombotic disease, viral infection, bacterial infection, and any combination thereof. In some embodiments, the pro-inflammatory disease comprises arthritis, Crohn's disease, psoriasis, and any combination thereof. In some embodiments, the viral infection is a SARS-CoV-2 infection, influenza, or any combination thereof. In some embodiments, the pro-thrombotic disease comprises deep vein thrombosis, pulmonary embolism, and any combination thereof. In some embodiments, the inhibitor is a pharmacologic agent. In some embodiments, the pharmacologic agent is a small molecule. In some embodiments, the small molecule comprises a chemical compound. In some embodiments, the inhibitor comprises forskolin, IBMX, SB239, tubacin, SB415286, or any combination thereof. In some embodiments, the inhibitor comprises, forskolin, IBMX, SB239, tubacin, or any combination thereof 16. The method of any one of claims 1 to 10, wherein the inhibitor comprises, forskolin, IBMX, SB239063, tubacin, SB431542, or any combination thereof. In some embodiments, the inhibitor comprises, forskolin, IBMX, SB239063, tubacin, SB431542, SB415286, or any combination thereof. In some embodiments, the inhibitor comprises, forskolin, IBMX, SB SB239063239, tubacin, K02288, or any combination thereof. In some embodiments, the inhibitor comprises, forskolin, IBMX, SB239063, tubacin, K02288, SB415286, or any combination thereof. In some embodiments, the inhibitor comprises, forskolin, IBMX, SB239063, tubacin, TCS-HDAC6, tubastatin, or any combination thereof. In some embodiments, the inhibitor comprises, forskolin, IBMX, SB239063, tubacin, TCS-HDAC6, tubastatin, SB415286, or any combination thereof.

In some embodiments, the inhibitor comprises an antibody. In some embodiments, the antibody comprises a neutralizing antibody. In some embodiments, the antibody comprises an antibody directed to IL-10, TNFα, or any combination thereof. In some embodiments, the inhibitor is an inhibitor of thrombin generation. In some embodiments, the inhibitor of thrombin generation is a Factor Xa inhibitor, a PAR1 inhibitor, or any combination thereof. In some embodiments, the inhibitor comprises a pharmacologic agent, an antibody, an inhibitor of thrombin generation, or any combination thereof.

In some embodiments, presented herein are pharmaceutical compositions comprising an inhibitor of capillary regression. In some embodiments, the capillary is a blood-carrying capillary, a lymphatic capillary, or any combination thereof. In some embodiments, the inhibitor of the pharmaceutical composition is a pharmacologic agent. In some embodiments, the pharmacologic agent is a small molecule. In some embodiments, the small molecule comprises a chemical compound. In some embodiments, the inhibitor comprises forskolin, IBMX, SB239, tubacin, SB415286, an inhibitor of thrombin generation, or any combination thereof. In some embodiments, the inhibitor of thrombin generation comprises a Factor Xa inhibitor, a PAR1 inhibitor, or any combination thereof. In some embodiments, the inhibitor comprises forskolin, IBMX, SB239063, tubacin, SB415286, or any combination thereof. In some embodiments, the inhibitor comprises forskolin, IBMX, SB239063, tubacin, or any combination thereof. In some embodiments, the inhibitor comprises, forskolin, IBMX, SB239063, tubacin, SB431542, or any combination thereof. In some embodiments, the inhibitor comprises, forskolin, IBMX, SB239063, tubacin, SB431542, SB415286, or any combination thereof. In some embodiments, the inhibitor comprises, forskolin, IBMX, SB239063, tubacin, K02288, or any combination thereof. In some embodiments, the inhibitor comprises, forskolin, IBMX, SB239063, tubacin, K02288, SB415286, or any combination thereof. In some embodiments, the inhibitor comprises, forskolin, IBMX, SB239063, tubacin, TCS-HDAC6, tubastatin, or any combination thereof. In some embodiments, the inhibitor comprises, forskolin, IBMX, SB239063, tubacin, TCS-HDAC6, tubastatin, SB415286, or any combination thereof. In some embodiments, the inhibitor comprises an antibody. In some embodiments, the antibody comprises a neutralizing antibody. In some embodiments, the antibody comprises an antibody directed to IL-10, TNFα, or any combination thereof.

In some embodiments presented herein are pharmaceutical compositions comprising a pharmacologic agent and an antibody. In some embodiments, the pharmacologic agent comprises forskolin, IBMX, SB239063, tubacin, SB415286, an ALK inhibitor, an inhibitor of thrombin generation, or any combination thereof. In some embodiments, the antibody comprises an antibody directed to IL-10, TNFα, or any combination thereof.

In some embodiments, presented herein are pharmaceutical compositions for use in the treatment of a disease in a patient in need thereof. In some embodiments, the pharmaceutical compositions comprise an inhibitor of capillary tube regression. In some embodiments, the pharmaceutical compositions comprise a therapeutically effective amount of the inhibitor of capillary tube regression. In some embodiments, the pharmaceutical compositions comprise an inhibitor of lymphatic tube regression. In some embodiments, the pharmaceutical compositions comprise a therapeutically effective amount of the inhibitor of lymphatic tube regression. In some embodiments, presented herein are pharmaceutical compositions for use in the treatment of a pro-inflammatory disease in a patient in need thereof. In some embodiments, presented herein are pharmaceutical compositions for use in the treatment of a pro-thrombotic disease in a patient in need thereof. In some embodiments, presented herein are pharmaceutical compositions for use in the treatment of a viral infection in a patient in need thereof. In some embodiments, presented herein are pharmaceutical compositions for use in the treatment of a bacterial infection in a patient in need thereof. In some embodiments, presented herein are pharmaceutical compositions for use in the treatment of COVID-19 in a patient in need thereof.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows exemplary IL-1 isoforms, TNFα and thrombin, singly and in combination, markedly induce EC tube regression. (A) EC cultures with tube networks were established for 48 hr followed by the addition of indicated cytokines or thrombin (Thr) for 24 hr. IL-1β and IL-la were added at 1 ng/ml, TNFα at 3.16 ng/ml and thrombin at 1 μg/ml. Bars equal 100 or 200 μm. (B,C,D) EC tube networks were treated with the indicated cytokines and varying concentrations in dose-response analyses and in the presence or absence of thrombin added at 1 μg/ml. After 24 hr, cultures were fixed, stained and quantitated for EC lumen area per field. These curves are from a representative experiment using triplicate wells for each condition. The values (±SD) are derived from analyzed fields (n=15) where total lumen and tube area was measured. Asterisks indicate significance at p<0.05 compared to control and squares indicate significance at p<0.05 compared to the cytokine treatments alone.

FIG. 2 shows exemplary toll-like receptor (TLR) activators induce macrophages to produce pro-regressive factors that appear similar to the pro-regressive abilities of IL-1 and TNFα. (A,B) The TLR ligand Pam3CSK4 was added at 100 ng/ml to THP-1 macrophages that were differentiated in two separate ways (THP-1-VT vs. THP-1-T) or to ECs, fibroblasts (Fib), or pericytes (Peri). Conditioned medium was collected after 4 hr and was added at a 1:5 dilution to established EC tube networks vs. control Pam3CSK4 or control RPMI1640 media. Two independent conditioned media batches were obtained for both the THP-1-VT (i.e. VT1, VT2) and THP-1-T (i.e. T1, T2) macrophage cells. IL-la and IL-1β were added separately at 1 ng/ml. After 24 hr, cultures were fixed, stained, photographed (A) and quantitated for EC tube area (B). Bar equals 200 μm. This data is derived from representative triplicate cultures. The values (±SD) were obtained from analyzed fields (n=15) where total lumen and tube area was measured. Asterisks indicate significance at p<0.05 compared to control. (C) LPS was added at the indicated concentrations (ng/ml) in the presence or absence of recombinant LPS binding protein (LBP) at 1 μg/ml to EC tube networks. EC lumen areas were measured after 24 hr following these additions. (D) THP-T macrophages were treated with control or 100 ng/ml of Pam3CSK4 (Pam) or LPS. After 4 hr, total RNA was obtained, and RT-PCR analysis was performed evaluating the mRNA expression of indicated genes. (E) Real-time video analysis coupled to measurements of EC tube regression over time. EC tube networks were treated with the indicated treatments including control, LPS-macrophage (1:50 dilution) and PAM-macrophage (1:20 dilution) media, TNFα (3.16 ng/ml), IL-1β (1 ng/ml) and IL-6 (40 ng/ml). From the real-time videos, EC lumen areas were measured over time (n=6).

FIG. 3 shows exemplary identification of the pro-regressive activities in TLR ligand-induced macrophage conditioned media as IL-1β and TNFα. (A,B) Pam3CSK4 or LPS induced macrophage conditioned were added to EC tube network cultures at a 1:20 or 1:50 dilution, respectively, in the presence or absence of neutralizing antibodies (10 μg/ml) to the indicated factors. Two different blocking antibodies directed to IL-6 were added, a mouse monoclonal antibody and the other a goat (gt) polyclonal antibody. In addition, FIST was added to control or macrophage media conditions. Cultures were fixed after 24 hr and tube area was quantitated. This data is derived from representative triplicate cultures. The values (±SD) were obtained from analyzed fields (n=15) where total lumen and tube area was measured. Asterisks indicate significance at p<0.05 compared to control. The triangle indicates increased significance relative to control, while the squares indicates significance relative to the Pam or LPS control regressed condition. (C-E) The indicated blocking antibodies, individually and in combination were added to Pam or LPS conditioned vs. control that were then added to EC tube network cultures. After 24 hr, cultures were quantitated for EC tube area (C,D) or were photographed (E). In addition, IL-1RA was added at 100 ng/ml in some conditions and FIST was added in separate conditions. Asterisks indicate significance at p<0.05 compared to control. The squares indicate significance relative to the Pam or LPS control regressed condition. Bar equals 200 μm.

FIG. 4 shows exemplary rescue of EC tube regression with FIST and FISTSB and identification of other growth factor and cytokines that can induce regression responses. (A) EC tube networks after 48 hr were treated with the indicated individual and combinations of factors and in the presence or absence of FIST or FISTSB. IL-1β was added at 1 ng/ml, TNFα at 3.16 ng/ml and thrombin at 1 mg/ml. All other growth factors and cytokines were added at 40 ng/ml. After 24 hr, cultures were fixed and quantitated for EC tube area. This data is derived from representative triplicate cultures. The values (±SD) were obtained from analyzed fields (n=15) where total lumen and tube area was measured. Asterisks indicate increased significance at p<0.05 compared to control, while the squares indicate decreased significance at p<0.05 relative to control. (B,C) EC tube networks were treated with the indicated factors and after 24 hr, tube area was quantitated. Asterisks indicate increased significance at p<0.05 compared to control.

FIG. 5 shows exemplary selective regression of EC tube networks, but not associated pericytes, during capillary regression caused by IL-1β, TNFα, thrombin and these factors in combination. (A) EC-pericyte (GFP-labeled) co-cultures were established and allowed to form for 72 hr and at this time, the combination of IL-1β (10 ng/ml), TNFα (10 ng/ml) and thrombin (11 μg/ml) were added in combination compared to control and real-time movies were performed. Light and fluorescent image overlays over time are shown in representative fields. Arrowheads indicate clusters of pericytes that accumulate together during regression responses in response to these three factors in combination. After 48 hr, fixed cultures were stained for CD31 to detect ECs and fluorescence image overlays were performed. (B) The same set up was established as in A, except that each individual factor and combination of factors were added. After 48 hr, the cultures were fixed and stained for CD31. Confocal images were obtained, and fluorescence overlay images are shown.

FIG. 6 shows exemplary defining of a capillary tube regression signaling signature during TNFα, IL-1β and thrombin induced regression responses. (A,B) ECs tube networks were treated with the indicated factors vs. control and at different time points, lysates were prepared from the 3D cultures. Western blots were performed using the different indicated antibodies. (C) Schematic illustrating findings that define a capillary regression signaling signature.

FIG. 7 shows exemplary comparable ability of IL-1 isoforms and TLR ligand induced macrophage conditioned media to induce regression of EC-lined tubes. (A) II-1β and IL-1α were added at 1 ng/ml to EC tube networks, and after 24 hr, cultures were fixed and photographed. (B) Pam3CSK4 was added to THP-1 cells differentiated by vitamin D3 and phorbol ester (THP-1-VT) vs. phorbol ester alone (THP-1-T) and conditioned media from these cells was added to EC tube networks in the presence or absence of FIST for 24 hr. Cultures, were fixed, stained and photographed. Bar equals 200 (C) EC-pericyte co-cultures were established for 72 hr and then control media, a Pam3CSK4 control, and then medium from the THP-1 cells described in B, was added for 24 hr. After fixation, cultures were stained with CD31 antibodies and overlay immunofluorescent images are shown. Bar equals 100 μm.

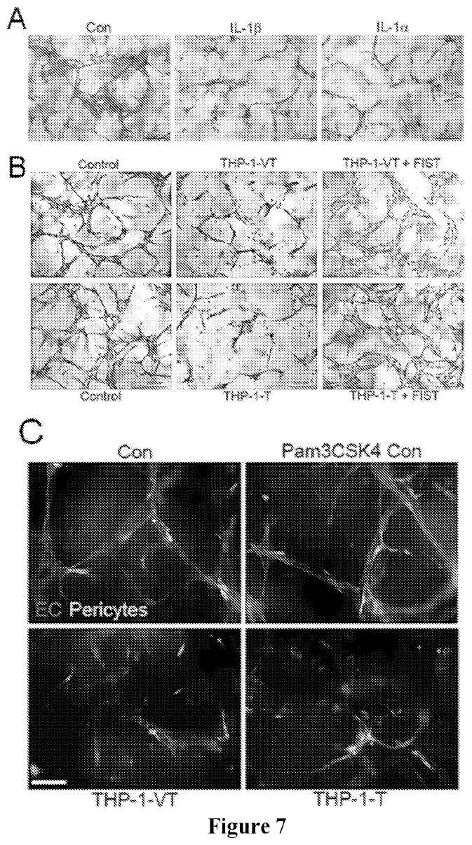

FIG. 8 shows exemplary addition of a Pak2 inhibitor and a microtubule depolymerizing agent mimic the pro-regressive activity of IL-1β and TNFα. (A) ECs were seeded onto a monolayer surface and then allowed to invade into the gels to form tubes over a 48 hr period. At this time, cultures were then treated with IL-1β or TNFα and in the absence or presence of FIST. Cultures were fixed after 24 hr, stained and photographed to visualize the monolayer surface and the underlying sprouts and tubes. (B) Cultures were established just like in A and then the Pak2 inhibitor, Frax486 (2.5 μM) or the microtubule depolymerizing agent, vinblastine (100 nM), were added to media in the presence or absence of FIST. Cultures were fixed after 24 hr, stained and photographed. Bar equals 200 μm.

FIG. 9 shows exemplary combinations and higher doses of IL-1β, TNFα, and thrombin induce marked capillary tube regression which can be rescued best with FISTSB: These pro-regressive factors also markedly interfere with the formation of EC tube networks. (A,B) EC tube networks were established and then thrombin alone was added (A) or combinations of IL-1β, TNFα and thrombin were added at two different doses and in the presence of FIST, FISTSB or FISTSBY (B), which added another drug, Y27632, a Rho kinase inhibitor. Addition of Y27632 did not enhance the ability of FISTSB to rescue capillary regression. TNFα was added at either 10 or 3.16 ng/ml, IL-10 at 10 or 1 ng/ml and thrombin was added at 1 μg/ml. Asterisks indicate increased statistical significance at p<0.05 and the squares indicate decreased statistical significance at p<0.05 compared to control. (C,D) EC cultures were set-up in the presence of the indicated factors, singly and in combination. IL-1β and TNFα were added at 10 ng/ml and thrombin was added at 1 μg/ml. Cultures were fixed after 72 hr, stained, photographed and quantitated for EC tube area. Asterisks indicate increased statistical significance at p<0.05.

FIG. 10 shows exemplary rescue of EC tube regression by FISTSB and FIST in response to combinations of IL-1β, TNFα, and thrombin. EC tube networks were treated with control, and with the combination of IL-1β and TNFα in the absence of presence of thrombin. Also, with each condition, FIST or FISTSB were added in comparison to control. Cultures were fixed after 24 hr, stained and photographed. Bar equals 200 μm.

FIG. 11 shows exemplary IL-1β, TNFα and thrombin induce selective regression of EC-lined tubes without loss of associated pericytes during capillary regression. (A) EC-pericyte co-cultures were established for 72 hr, and then were left untreated or with IL-1β (10 ng/ml), TNFα (10 ng/ml), and thrombin (11 μg/ml) in combination. After 48 hr, cultures were fixed and stained with CD31, confocal microscopy and 3D reconstruction of the images allowed for an upright and side view. (B) EC-pericyte tube networks were treated with the indicated single and combined factors and after fixation, EC tube area was quantitated. This data is derived from representative triplicate cultures. The values (±SD) were obtained from analyzed fields (n=15) where total lumen and tube area was measured. Asterisks indicate increased significance at p<0.05 compared to control, the squares indicate decreased significance at p<0.05 relative to the single factor conditions and the triangle indicates significance at p<0.05 relative to the double factor conditions. (C) EC-pericyte tube networks were treated with indicated combinations of factors. After 48 hr, total GFP fluorescence per well was quantitated as a measure of pericyte number since they are GFP-labeled. This data is derived from representative triplicate cultures. Asterisks indicate significance at p<0.05 compared to control.

FIG. 12 shows exemplary TLR-treated macrophage conditioned media induce a capillary regression signaling signature during EC tube regression. THP-T macrophages were treated with Pam3CSK4 (Pam) or LPS and conditioned medium was added at a 1:20 or 1:50 dilution, respectively, to EC tube networks. Control and IL10-treated cultures were compared with the conditioned medium treatments within the same experiment. At the indicated time points, lysates were obtained, and Western blots were performed with the indicated antibodies.

FIG. 13 shows a schematic illustrating pro-inflammatory stimuli that can lead to capillary loss and local or systemic pathological effects. Therapeutic options to block capillary loss are also illustrated.

FIG. 14 shows pharmacologic rescue of capillary regression induced by combinations of proinflammatory mediators. EC tube networks were established for 48 hr and the indicated mediators were added for 24 hr. Each of the mediators was added at 10 ng/ml, except for thrombin (Thr) which was added at 1 μg/ml. The indicated combinations of mediators were added in the absence or presence of the indicated combinations of pharmacologic agents. FISTSB=F-forskolin-10 μM; I-IBMX-100 μM; S-SB239063-10 μM; SB-SB415286-25 μM; T-TCS-HDAC6-10 μM; FISTSB2=FISTSB+SB431542-10 μM; K-K02288-10 μM. Cultures were fixed, stained with toluidine blue and photographed.

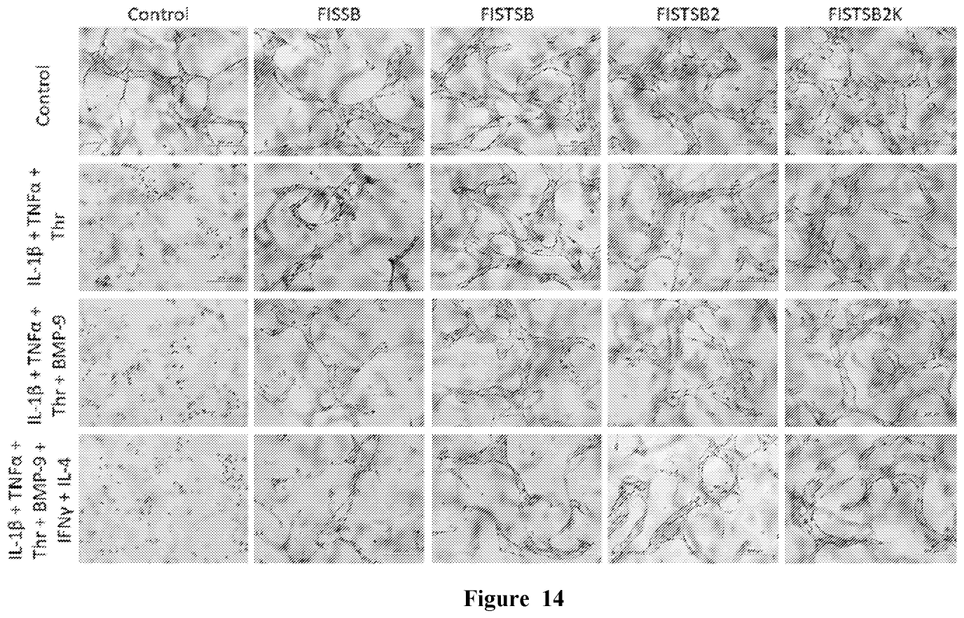

FIG. 15 shows complete pharmacologic rescue of capillary regression induced by combinations of pro-inflammatory mediators. EC tube networks were established for 48 hr and the indicated mediators were added for 24 hr. Each of the mediators was added at 10 ng/ml, except for thrombin (Thr) which was added at 1 μgn/ml. HD=IL-10+TNFα+Thrombin. The indicated combinations of mediators were added in the absence or presence of the indicated combinations of pharmacologic agents. F-forskolin-10 μM; I-IBMX-100 μM; S-SB239063-10 μM; SB-SB415286-25 μM; TTCS-HDAC6-10 μM; FISTSB2=FISTSB+SB431542-10 μM; K-K02288-10 μM. Cultures were fixed, stained with toluidine blue, photographed, and quantitated for EC lumen area.

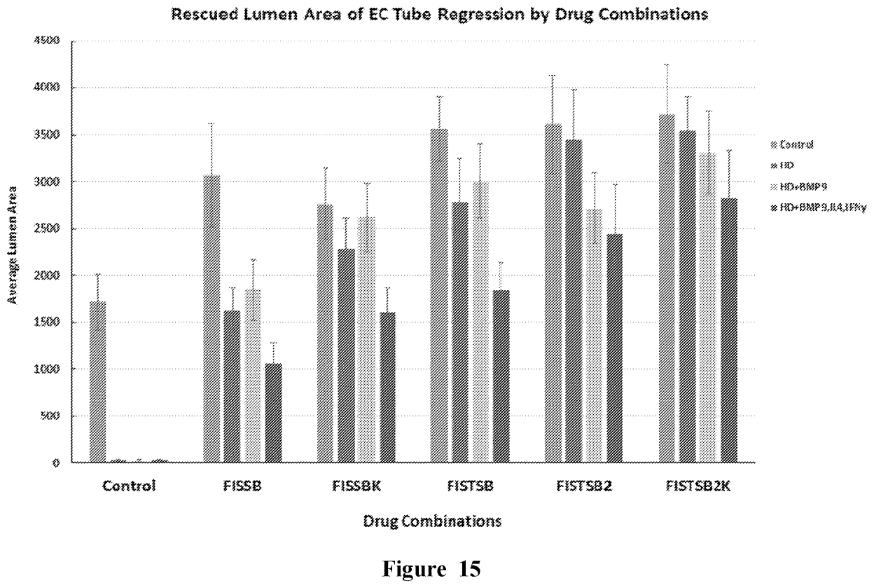

FIG. 16 shows addition of different HDAC6 inhibitors, TCS-HDAC6, tubacin, and tubastatin enhance the pharmacologic rescue of capillary regression induced by pro-inflammatory mediators. EC tube networks were established for 48 hr and the indicated mediators were added for 24 hr. Each of the mediators was added at 10 ng/ml, except for thrombin (Thr) which was added at 1 μg/ml. The indicated combinations of mediators were added in the absence or presence of the indicated combinations of pharmacologic agents. FISSB2=F-forskolin-10 μM; IMMX-100 μM; S-SB239063-10 μM; SB-SB415286-25 μM; and SB431542-10 μM. FISSB2 was added where indicated by itself or in the presence of the HDAC6 inhibitors, TCS-HDAC6-10 μM; Tubacin-10 μM or Tubastatin-10 μM. Cultures were fixed, stained with toluidine blue and photographed.

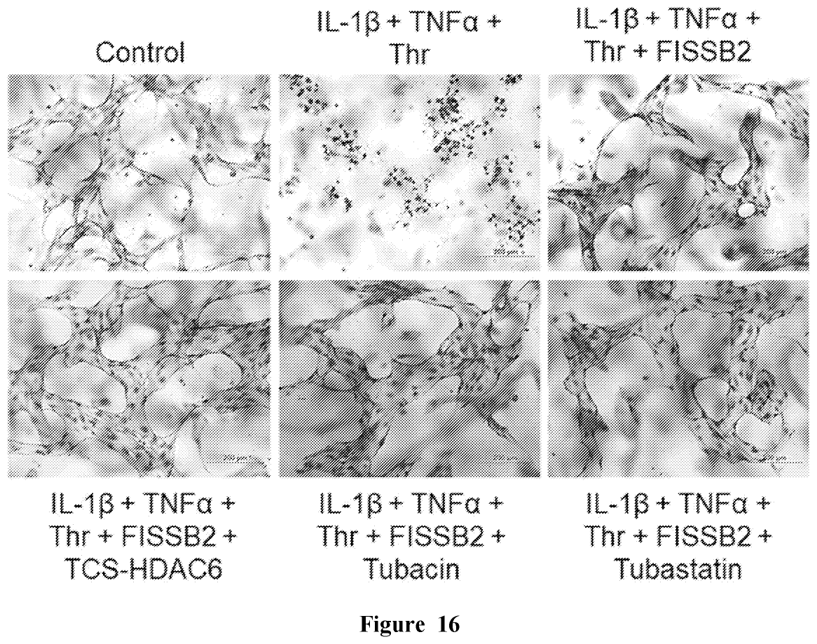

FIG. 17 shows lymphatic EC-lined tube networks regress in the presence of the proinflammatory mediators, TNFα and thrombin. Lymphatic EC tube networks were established for 48 hr and at that time, the indicated pro-inflammatory mediators were added. IL-10 and TNFα were added at 10 ng/ml, while thrombin was added at 1 μg/ml. (A) After 24 hr, cultures were fixed, stained with toluidine blue and photographed. (B) The cultures in (A) were quantitated for EC tube area. Asterisks indicate significance at p<0.01 compared to control.

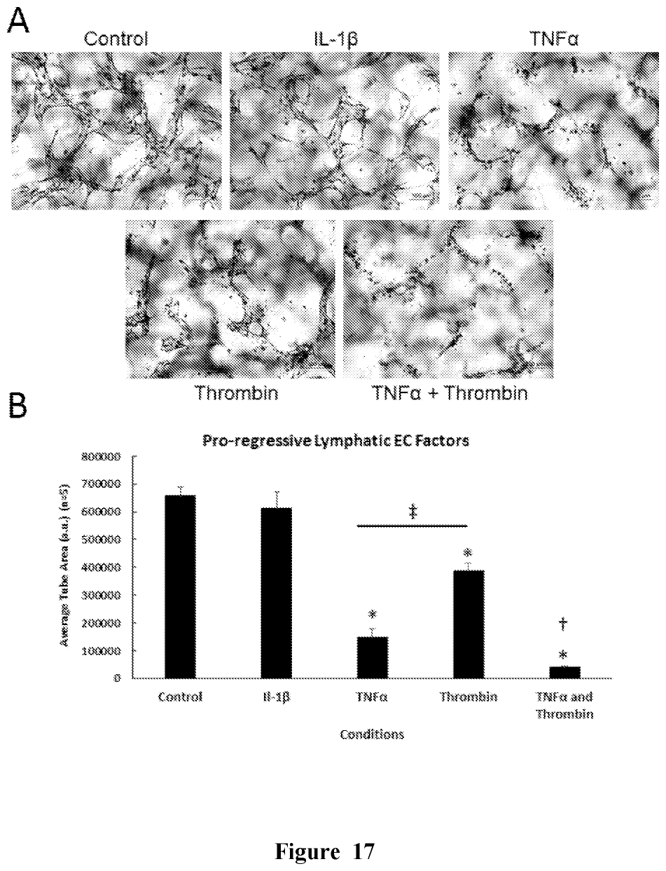

FIG. 18 shows evidence for a proinflammatory macrophage environment, role for IL (induced)-1β and TNF (tumor necrosis factor) a, and the presence of regression signaling signature during hyaloid vascular regression in mice. (A) Intravitreal injection of blocking antibodies directed to both IL-10 and TNFα vs control IgG was performed at postnatal day (P) 6, and hyaloid vessel number was quantitated at P8. (B) Intravitreal injection of IL-10 was performed at P5 vs vehicle control, and hyaloid vessel number was quantitated at P8. (C and D) Hyaloid vessels at P2 and P8 were immunostained for phospho-MLC2 (p-MLC; red) and CD31-positive endothelial cells (ECs; green), and double-positive cells were imaged and quantitated over time. Scale bar=100 μm. E and F, Analysis of ICAM-1 expression at the mRNA (E) and protein level (F) during different stages of hyaloid vessel regression. CD indicates cluster of differentiation; DAPI, 4′6-diamidino-2-phenylindole; ICAM, intercellular adhesion molecule; and MLC, myosin light chain.

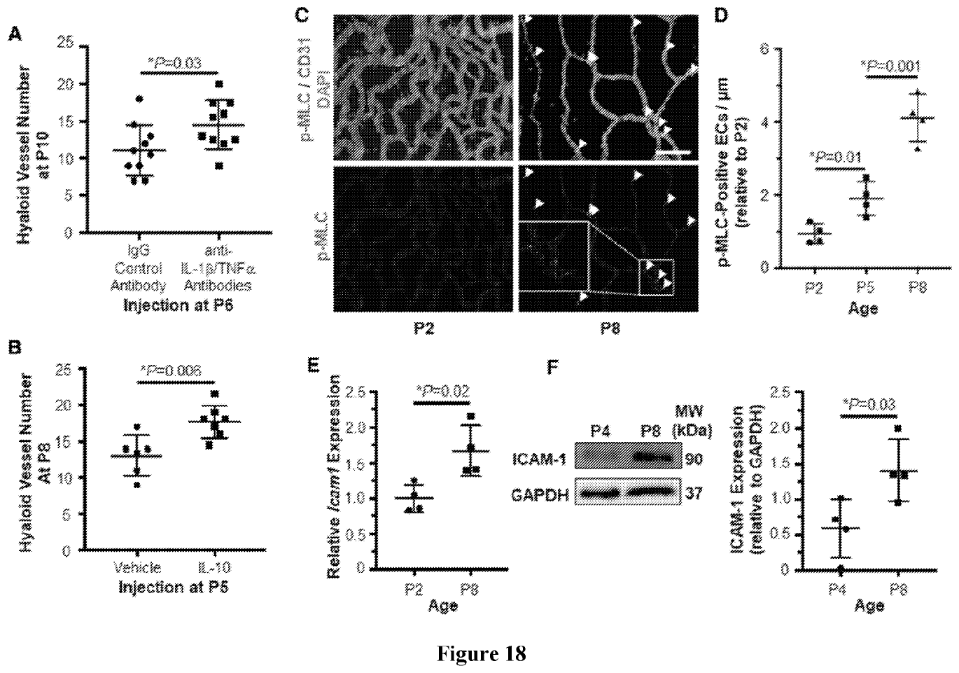

FIG. 19 shows hyaloid vascular regression is accompanied by increased pro-inflammatory cytokine expression during the regression response. (A) Immunostaining of the hyaloid vasculature at the indicated time points. Scale bar=500 μm. (B) Quantitation of hyaloid vessel regression over time. (C) Immunostaining revealing F4/80+ macrophage association with regressing hyaloid vessels. Note, Isolectin-B4 marks both hyaloid vessels and macrophages. (D) Immunostaining showing increased IL-1b expression (white arrowheads) in Iba-1+ macrophages associated with regressing hyaloid vessels. Scale bar=50 μm. (E,F,G) qPCR analysis of IL-1 and TNF transcript expression during hyaloid vessel regression. Statistical significance is indicated relative to other time points during hyaloid regression.

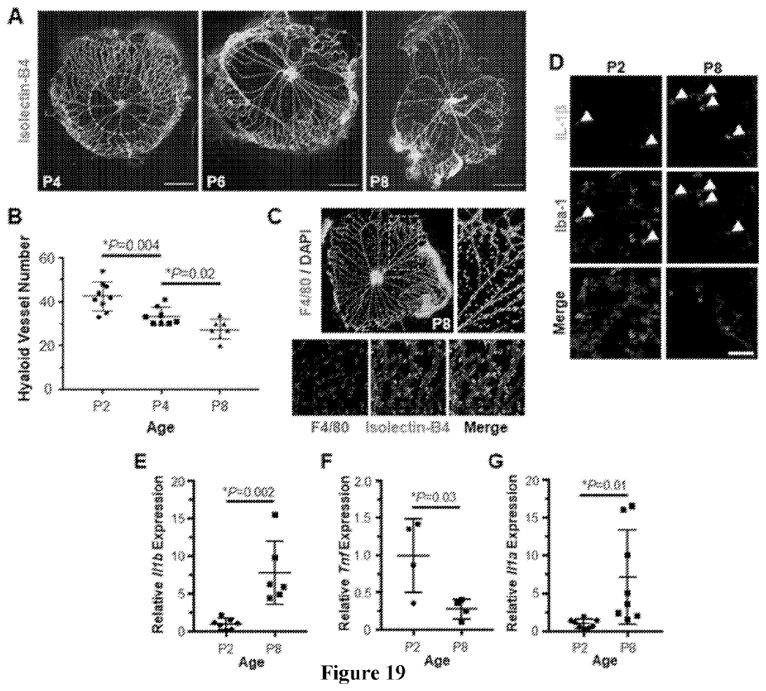

### DETAILED DESCRIPTION

Detailed herein are compositions and methods of treatment for inhibiting capillary tube regression and/or lymphatic tube network regression. In some embodiments, factors and signaling pathways that control the regression of capillary tube networks are also described herein. In some embodiments, a detailed screen of growth factors, and cytokines as well as the inflammatory mediator, thrombin, can be used to identify IL-1, TNFα and thrombin (and these mediators in combination), as markedly causing endothelial cell (EC) tube collapse and EC apoptosis over a period of time. In some embodiments, this period of time is between about 24 to about 48 hours. In some embodiments, these pro-regressive mediators selectively induce the regression of EC-lined tube networks with EC cell death, while associated pericytes are spared and proliferate in response. In some embodiments, pro-inflammatory macrophages which can be activated by the TLR agonists, Pam3CSK4 or LPS, produced potent pro-regressive activities. In some embodiments, these pro-regressive activities were identified as IL-1β and TNFα. In some embodiments, other pro-regressive factors were identified. In some embodiments, these other pro-regressive activities had a potency level at the potency level of IL-1β and TNFα (e.g., IL-la). In some embodiments, these other pro-regressive activities had a potency level below the potency level of IL-10 and TNFα. In some embodiments, thrombin, which is often co-generated in tissue injury responses along with cytokines such as IL-10 and TNFα, further enhances the pro-regressive activity of these cytokines individually or when they are combined. In some embodiments, the pro-inflammatory cytokines, IL-10 and TNFα, in conjunction with thrombin are the major factors that promote capillary tube regression. In some embodiments, the pro-inflammatory cytokines, IL-1β, IL-la, TNFα, in conjunction with thrombin are the major factors that promote capillary tube regression. In some embodiments, thrombin enhances the pro-regressive activities of both IL-1 isoforms and TNFα. In some embodiments, in serious tissue injury contexts, all three of these pro-regressive molecules will be present (e.g., in a “cytokine storm”), and thus, it can be necessary to inhibit them in combination to interfere with capillary regression responses. In some embodiments, capillary regression can be a pathogenic factor in human diseases. Non-limiting examples of human diseases where capillary regression can be a pathogenic factor can include ischemia and infarction, diabetes, hypertension, neurodegenerative diseases, malignant cancers, heart failure, sepsis, coronavirus disease 2019 (COVID-19), acute respiratory distress syndrome (ARDS), a viral infection or viral infectious disease, and a bacterial infection or bacterial infectious disease. In some embodiments, the viral infection is a SARS-CoV-2 infection, a coronavirus infection, a rhinovirus infection, an adenovirus infection. In some embodiments, the viral infectious disease is influenza, COVID-19, or pneumonia. In some embodiments, neutralizing antibodies to TNFα and IL-1β as well as chemical inhibitors that block thrombin generation (e.g., Factor Xa inhibitors and/or protease-activated receptor 1 (PAR1) inhibitors) can provide a therapeutic strategy in these clinical situations.

In some embodiments, in severe cases, COVID-19 is associated with acute respiratory distress syndrome (ARDS), which classically results from increased permeability of pulmonary capillaries. In some embodiments, the vascular leakage characteristic of ARDS diminishes the capacity for oxygen to diffuse from the alveolar space into adjacent capillaries, leading to respiratory failure and systemic hypoxia. In some embodiments, other mechanisms that can contribute to capillary dysfunction and hypoxia include development of microthrombi and endothelial cell (EC) death. In some embodiments, COVID-19 patients exhibit pulmonary capillary damage, including microthrombi, inflammatory cell infiltration into small vessels, EC death, edema, and focal hemorrhages. In some embodiments, COVID-19 might lead to EC death and capillary regression in lungs and other organs, resulting in an ARDS-like condition, systemic hypoxia and multi-organ failure.

In some embodiments, patients with particularly severe COVID-19 symptoms frequently show marked elevation of pro-inflammatory factors such as thrombin, interferon gamma (IFNγ), and the cytokines TNFα, IL-6, and IL-1β. In some embodiments, thrombin, IFNγ, TNFα, IL-6, and IL-1β can cause vascular permeability and/or thrombosis in a COVID-19 patient. In some embodiments, thrombin, IFNγ, TNFα, IL-6, IL-1β, and any combination thereof can cause vascular damage in a COVID-19 patient. In some embodiments, IFNγ and interleukin-4 (IL-4) can have a strong pro-regressive activity. In some embodiments, IFNγ and IL-4 can be upregulated factors in the “cytokine storm” exhibited by COVID-19 patients.

In some embodiments, the severe acute respiratory syndrome coronavirus 2 (SARS-CoV-2) can induce capillary regression in infected tissues such as the lung and heart, which are essential for systemic oxygenation and tissue health. In some embodiments, macrophages are a major source of inflammatory cytokines, including the ones that can trigger capillary regression. In some embodiments, SARS-coronaviruses and other highly pathogenic viruses can activate the NLRP3 inflammasome, which leads to production and release of IL-1β and IL-18 from various cell types, including macrophages. Therefore, in some embodiments, direct infection of macrophages with SARS-CoV-2, or interactions between macrophages and other infected cell types (e.g., in the alveolar wall), can induce inflammasome activation and local elevation of active IL-10 as well as upregulate the production and release of other key mediators such as TNFα and thrombin that could drive capillary regression.

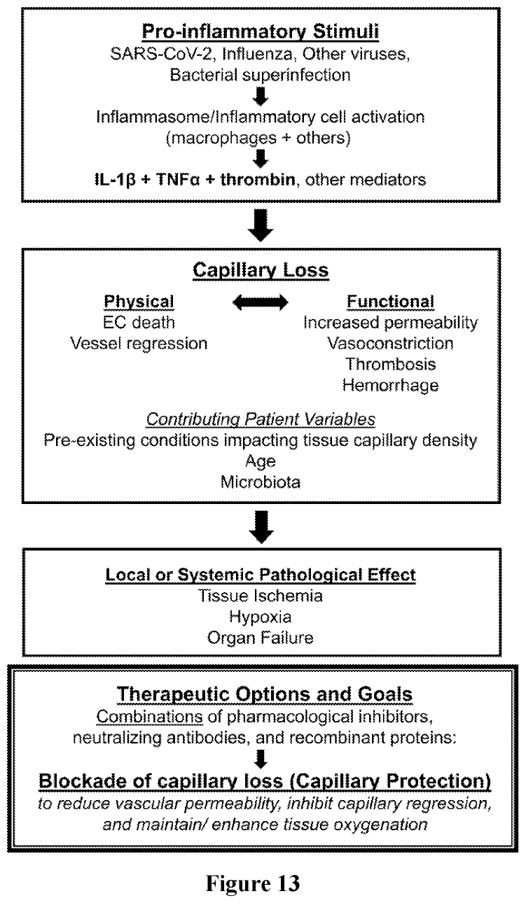

In some embodiments, identifying a capillary regression signaling signature can help determine if capillary regression is occurring in COVID-19 patients. In some embodiments, the capillary regression signaling signature includes increased levels of phospho-p38 Map kinase, phospho-MLC2, and/or an induction of ICAM-1 compared to basal levels (e.g., of a healthy patient or an uninfected patient). In some embodiments, the capillary regression signaling signature includes decreased levels of phospho-Pak2, phospho-cofilin, acetylated tubulin, and/or pro-caspase3 compared to basal levels (e.g., of a healthy patient or an uninfected patient). In some embodiments, the severity of the COVID-19 in a patient can relate to the extent of capillary loss during the infection coupled to the basal state of pulmonary capillary density and function that was present in the patient prior to viral infection, as shown in FIG. 13.

In some embodiments, the combination of TNFα and thrombin, but not IL-1β, can cause marked regression of lymphatic tube networks. In some embodiments, pro-inflammatory mediators can cause regression of blood-carrying capillary tubes, and also regression of lymphatic tube networks. In some embodiments, these latter lymphatic networks remove pro-inflammatory mediators from tissue injury sites. Thus, in some embodiments, these mediators can persist in these injured sites due to loss of lymphatic capillaries, which might further induce blood-carrying capillary regression. In some embodiments, the development of capillary protection agents can be used to prevent the regression of both blood containing capillaries as well as lymphatic capillaries.

In some embodiments, IL-1β, IL-la, and TNFα can be potent regulators of capillary tube regression. In some embodiments, pro-inflammatory macrophages can produce pro-regressive factors for capillary networks. In some embodiments, macrophages (e.g., human macrophages) exposed to TLR ligands can induce pro-regressive activity as compared to a non-exposed control. Non-limiting examples of TLR ligands can include Pam3CSK4 and LPS. In some embodiments, Pam3CSK4 and LPS are added to the macrophages in combination at the same time. In some embodiments, Pam3CSK4 and LPS are added to the macrophages in combination at different times. In some embodiments, addition of either TLR ligand itself to EC tube networks does not influence capillary regression. In some embodiments, Pam3CSK4- and LPS-induced macrophage media pro-regressive activity can be blocked by a combination of antibodies directed to both IL-1β and TNFα. In some embodiments, neutralizing antibodies directed to other less potent pro-regressive growth factors and cytokines do not block the influence of the macrophage conditioned medium.

In some embodiments, physiologic capillary regression can be modeled in a mouse by examining the postnatal regression of the eye hyaloid vasculature. In some embodiments, this vascular regressive response can depend on the presence of adjacent macrophages which decorate this developmental vascular bed. In some embodiments, intraocular administration of blocking antibodies to TNFα and IL-1β in combination, as compared to a vehicle control, reduced the hyaloid regression response. In some embodiments, injection of the anti-inflammatory cytokine, IL-10 (which is known to decrease TNFα and IL-1β expression), also inhibited hyaloid regression. In some embodiments, EC capillary regression during hyaloid regression can include increased expression of ICAM-1 (at the mRNA and protein level) and increased presence of phospho-MLC2 in regressing hyaloid ECs.

In some embodiments, IL-1β, TNFα, and thrombin, selectively induce EC tube regression but pericytes, despite their assembly on the abluminal surface of the regressing tubes, remain intact and proliferate in response to the pro-regressive factors. In some embodiments, pericytes do not decline and regress under EC tube regression by IL-1β, TNFα, and thrombin. In some embodiments, cellular pathways activated during regression induced with IL-1β and TNFα treatment as well as with macrophage conditioned medium can include increased phospho-p38 Map kinase, phospho-MLC2, and induction of ICAM-1. In some embodiments, pathways that are inactivated during regression induced with IL-1β and TNFα treatment as well as with macrophage conditioned medium can include decreased levels of phospho-Pak2, phospho-cofilin, acetylated tubulin and procaspase3. In some embodiments, the activation is accentuated when thrombin is added to IL-1β or TNFα. In some embodiments, the inactivation is accentuated when thrombin is added to IL-1β or TNFα.

In some embodiments, pharmacologic agents (e.g., inhibitors or agonists) can prevent EC tube regression events. In some embodiments, pharmacologic agents (e.g., inhibitors or agonists) can prevent lymphatic tube regression events. In some embodiments, pharmacologic agents (e.g., inhibitors or agonists) can prevent EC tube regression events, EC death, and/or lymphatic tube regression occurring in an infected patient (e.g., infected by SARS-CoV-2, influenza, or bacteria). In some embodiments, pharmacologic agents (e.g., inhibitors or agonists) can prevent EC tube regression events, EC death, and/or lymphatic tube regression caused by an infection (e.g., a SARS-CoV-2 infection, influenza, or bacterial infection). In some embodiments, pharmacologic agent combinations can rescue back to control levels or above the EC tube regression, EC death, and/or lymphatic tube regression events induced by the pro-regressive factors and their combinations described herein. In some embodiments, a reduction of EC tube regression by about 100%, about 98%, about 96%, about 94%, about 92%, about 90%, about 88%, about 86%, about 84%, about 82%, about 80%, about 75%, about 70%, about 65%, about 60%, about 55%, about 50%, or any value between indicates rescue of the pro-regressive factors and there combinations.

In some embodiments, the pharmacologic agent combination can include agents involved in stimulating cyclic AMP levels, inhibiting p38 Map kinases, inhibiting tubulin or histone deacetylase (e.g., HDAC), and inhibiting GSK3(3. In some embodiments, the pharmacologic agent combination can include FIST (e.g., forskolin, IBMX (3-isobutyl-1-methylxanthine), SB239, tubacin). In some embodiments, SB239 is sometimes referred to SB239063. In some embodiments, the pharmacologic agent combination can include FISTSB (e.g., forskolin, IBMX, SB239, tubacin, SB415286). In some embodiments, the capillary regression can have a common signaling mechanism. In some embodiments, FISTSB is more effective, as compared to FIST, at rescuing these factors when they are added in combination. In some embodiments, FISTSB is more effective, as compared to FIST, at rescuing the combinations of IL-1β or TNFα with thrombin. In some embodiments, FISTSB is more effective, as compared to FIST, at rescuing high concentrations of IL-10 and TNFα when added individually, in combination or with thrombin. In some embodiments, the high concentration of IL-10 and TNFα when added individually, in combination or with thrombin can mimic what can occur in serious tissue injury and life-threatening situations where systemic macrophage activation and vascular permeability occur together leading to high levels of IL-1β, TNFα and thrombin. In some embodiments, a high concentration is greater than 1 ng/ml. In some embodiments, a high concentration is greater than 3 ng/ml. In some embodiments, a high concentration is approximately 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, ng/ml, 30 ng/ml, 40 ng/ml, 50 ng/ml, 60 ng/ml, 70 ng/ml, 80 ng/ml, 90 ng/ml, 100 ng/ml, or greater. In some embodiments, a high concentration is approximately 10 ng/ml.

In some embodiments, the pharmacologic agent can include an antagonistic recombinant protein that inhibits one or more of the pro-regressive inflammatory mediators disclosed herein. In some embodiments, the antagonistic recombinant protein is interleukin-1 receptor antagonist (IL-1RA). In some embodiments, the pharmacologic agent can include a histone deacetylase (HDAC) inhibitor. In some embodiments, the HDAC inhibitor is a class I, IIA, IIB, III, or IV HDAC inhibitor. In some embodiments, the HDAC inhibitor includes tubastatin, TCS-HDAC6, tubacin, and any combinations thereof. In some embodiments, the pharmacologic agent can include activin receptor-like kinase 4 (ALK4) and/or ALK5 inhibitor. In some embodiments, the pharmacologic agent can include SB431542. In some embodiments, the pharmacologic agent can include ALK2 and/or ALK1 inhibitor. In some embodiments, the pharmacologic agent can include K02288.

In some embodiments, EC capillary regression and/or lymphatic tube regression can be rescued by a combination of pharmacologic agents. In some embodiments, EC capillary regression and/or lymphatic tube regression occurring in an infected patient (e.g., infected by SARS-CoV-2, influenza, or bacteria) can be rescued by a combination of pharmacologic agents. In some embodiments, EC capillary regression and/or lymphatic tube regression caused by an infection (e.g., a SARS-CoV-2, influenza, or bacterial infection) can be rescued by a combination of pharmacologic agents. In some embodiments, the combination of pharmacologic agents can include FIST, FISTSB, an antagonistic recombinant protein, or combinations thereof. In some embodiments, the combination of pharmacologic agents can include FIST, FISTSB, IL-1RA, or combinations thereof. In some embodiments, the combination of pharmacologic agents can include FIST, FISTSB, an HDAC inhibitor, or combinations thereof. In some embodiments, the combination of pharmacologic agents can include FIST, FISTSB, tubastatin, TCS-HDAC6, tubacin, or combinations thereof.

Activin receptor-like kinases (ALKs) are receptors in the transforming growth factor beta (TGF-β) signaling pathway. In some embodiments, TGF-β can induce EC tube regression. In some embodiments, TGF-β is a pro-regressive factor. In some embodiments, inhibiting TGF-β, disrupting the TGF-β signaling pathway, or inhibiting an ALK (e.g., ALK4, ALK5, ALK1, and/or ALK2) can reduce, reverse, or prevent EC tube regression. In some embodiments, the combination of pharmacologic agents can include FIST, FISTSB, an ALK4 inhibitor, an ALK5 inhibitor, or combinations thereof. In some embodiments, the combination of pharmacologic agents can include FIST, FIRSTSB, SB431542, or combinations thereof. In some embodiments, the combination of pharmacologic agents can include FIST, FISTSB, an ALK2 inhibitor, an ALK1 inhibitor, or combinations thereof. In some embodiments, the combination of pharmacologic agents can include FIST, FIRSTSB, K02288, or combinations thereof.

In some embodiments, the combination of pharmacologic agents can include FIST, FIRSTSB, SB431542, K02288, or combinations thereof. In some embodiments, the combination of pharmacologic agents can include FIST, FIRSTSB, SB431542, K02288, tubastatin, TCS-HDAC6, tubacin, or combinations thereof.

In some embodiments, EC capillary regression and/or lymphatic tube regression can be rescued by a combination of pharmacologic agents and one or more antibodies. In some embodiments, EC capillary regression and/or lymphatic tube regression occurring in an infected patient (e.g., infected by SARS-CoV-2, influenza, or bacteria) can be rescued by a combination of pharmacologic agents and one or more antibodies. In some embodiments, EC capillary regression and/or lymphatic tube regression caused by an infection (e.g., a SARS-CoV-2, influenza, or bacterial infection) can be rescued by a combination of pharmacologic agents and one or more antibodies. In some embodiments, the one or more antibodies can include neutralizing antibodies. In some embodiments, the neutralizing antibodies can include antibodies directed to IL-1β, TNFα, IFNγ, IL-4, IL-13, or combinations thereof.

In some embodiments, the combination of pharmacologic agents can include an agent stimulating cyclic AMP levels, one or more antibodies (e.g., neutralizing antibodies), or any combinations thereof. In some embodiments, the combination of pharmacologic agents can include a p38 Map kinase inhibitor, one or more antibodies (e.g., neutralizing antibodies), or any combinations thereof. In some embodiments, the combination of pharmacologic agents can include an HDAC inhibitor, one or more antibodies (e.g., neutralizing antibodies), or any combinations thereof. In some embodiments, the combination of pharmacologic agents can include a GSK3β inhibitor, one or more antibodies (e.g., neutralizing antibodies), or any combinations thereof. In some embodiments, the combination of pharmacologic agents can include an ALK inhibitor (e.g., ALK4, ALK5, ALK2, and/or ALK1 inhibitors), one or more antibodies (e.g., neutralizing antibodies), or any combinations thereof. In some embodiments, the combination of pharmacologic agents can include an antagonistic recombinant protein (e.g., IL-1RA), one or more antibodies (e.g., neutralizing antibodies), or any combinations thereof. In some embodiments, the combination of pharmacologic agents can include a Factor Xa inhibitor, one or more antibodies (e.g., neutralizing antibodies), or any combinations thereof. In some embodiments, the combination of pharmacologic agents can include a protease activated receptor 1 (PAR1) inhibitor, one or more antibodies (e.g., neutralizing antibodies), or any combinations thereof.

In some embodiments, the combination of pharmacologic agents can include an agent stimulating cyclic AMP levels, a p38 Map kinase inhibitor, an HDAC inhibitor, a GSK3β inhibitor, an ALK inhibitor, an antagonistic recombinant protein, a Factor Xa inhibitor, a PAR1 inhibitor, one or more antibodies (e.g., neutralizing antibodies), or any combinations thereof. In some embodiments, the combination of pharmacologic agents can include FIST, FISTSB, an ALK inhibitor, an antagonistic recombinant protein, a Factor Xa inhibitor, a PAR1 inhibitor, one or more antibodies (e.g., neutralizing antibodies), or any combinations thereof.

In some embodiments, the combination of pharmacologic agents can include FIST, FISTSB, an ALK inhibitor (e.g., ALK4, ALK5, ALK2, and/or ALK1 inhibitors), one or more antibodies (e.g., neutralizing antibodies), or combinations thereof. In some embodiments, the combination of pharmacologic agents can include an antagonistic recombinant protein (e.g., IL-1RA), one or more antibodies (e.g., neutralizing antibodies), or combinations thereof. In some embodiments, the combination of pharmacologic agents can include a Factor Xa inhibitor, one or more antibodies (e.g., neutralizing antibodies), or combinations thereof. In some embodiments, the combination of pharmacologic agents can include a protease activated receptor 1 (PAR1) inhibitor, one or more antibodies (e.g., neutralizing antibodies), or combinations thereof.

In some embodiments, the combination of pharmacologic agents can include an agent stimulating cyclic AMP levels, a p38 Map kinase inhibitor, an HDAC inhibitor, a GSK3β inhibitor, an ALK inhibitor, an antagonistic recombinant protein, a Factor Xa inhibitor, a PAR1 inhibitor, one or more antibodies (e.g., neutralizing antibodies), or combinations thereof. In some embodiments, the combination of pharmacologic agents can include FIST, FISTSB, an ALK inhibitor, an antagonistic recombinant protein, a Factor Xa inhibitor, a PAR1 inhibitor, one or more antibodies (e.g., neutralizing antibodies), or any combinations thereof.

In some embodiments, the combination of pharmacologic agents can include FIST, FIRSTSB, SB431542, one or more antibodies (e.g., neutralizing antibodies), or any combinations thereof. In some embodiments, the combination of pharmacologic agents can include FIST, FIRSTSB, K02288, one or more antibodies (e.g., neutralizing antibodies), or any combinations thereof. In some embodiments, the combination of pharmacologic agents can include FIST, FIRSTSB, tubastatin, TCS-HDAC6, one or more antibodies (e.g., neutralizing antibodies), or any combinations thereof. In some embodiments, the combination of pharmacologic agents can include FIST, FIRSTSB, IL-1RA, one or more antibodies (e.g., neutralizing antibodies), or any combinations thereof. In some embodiments, the combination of pharmacologic agents can include FIST, FIRSTSB, SB431542, K02288, tubastatin, TCS-HDAC6, IL-1RA, a Factor Xa inhibitor, a PAR1 inhibitor, one or more antibodies (e.g., neutralizing antibodies), or any combinations thereof. In some embodiments, the pharmacologic agents described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

In some embodiments, the pharmacologic agents are administered at a dose from about 1 mg/kg to about 100 mg/kg. In some embodiments, the pharmacologic agents are administered at a dose at about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, or 200 mg/kg. In some embodiments, the dose is a therapeutically effective amount. In some embodiments, the pharmacologic agents can be administered on a daily basis (e.g., as a single dose or as two or more divided doses) or non-daily basis (e.g., every other day, every two days, every three days, once weekly, twice weeks, once every two weeks, once a month). In some embodiments, the period of administration of the pharmacologic agents as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Further embodiments of the disclosure can be found in the attached Appendix A which is herein incorporated by reference in its entirety. The disclosure is further described in the following non-limiting examples.

### EXAMPLES

### Methods

### Materials

Human umbilical vein endothelial cells (HUVECs) were from Lonza, human brain vascular pericytes from ScienCell and THP-1-Null cells from Invivogen. Culture media (e.g., Medium 199) were from Fisher Scientific and FCS was from Sigma. All biochemical reagents were from Tocris, except for Pam3CSK4 which were from Invivogen, phorbol ester from Sigma, and Huzzah KLA from Avanti-Polar Lipids. Neutralizing antibodies were from R&D Systems, and all Western blotting antibodies were from Cell Signaling Technology, except for acetylated tubulin antibodies from Millipore, and tubulin antibodies from Sigma.

### Cell Culture (HUVEC and THP-1)

ECs (passages 3-6) and pericytes (passages 3-12) were cultured as previously described^{49,50}. THP-1 cells were cultured in RPMI-1640-20% FCS. THP-1-T macrophages were differentiated for 72 hr. with 50 ng/ml of phorbol ester in RPMI-20% FCS, and then treated for 48 hr. in RPMI-2% FCS prior to adding TLR ligands. THP-1-VT macrophages were induced to differentiate for 72 hr. with 10-7 M vitamin D3 (Calcitriol) in RPMI-20% FCS, followed by 48 hr. with phorbol ester in RPMI-2% FCS prior to adding TLR ligands.

### Macrophage Activation Experiments/Timecourses

Huzzah-KLA (i.e. LPS) or Pam3CSK4 were added to THP-1 cells in culture at a final concentration of 100 ng/ml in RPMI-1640 containing the RSII serum-free supplement. Conditioned media or total RNA were collected after 4 hr. RT-PCR primer sets:

F3:
F-5′-tgtgaaggatgtgaagcagacg-3′;
R-5′-gggagttctccttccagctctg-3′;
IL-1α:
F-5′-accttcaaggagagcatggtgg-3′:
R-5′-ttggtcttcatcttgggcagtc-3′
IL-1β:
F-5′-cgatcactgaactgcacgctc-3′;
R-5′-ttatatcctggccgcctttgg-3′
TNFα:
F-5′-ttctccttcctgatcgtggcag-3′;
R-5′-tgatggcagagaggaggttgac-3′
IL-6:
F-5′-caaattcggtacatcctcgacg-3′;
R-5′-tgtcctgcagccactggttctg-3′
IL-10:
F-5′-atgcacagctcagcactgctc-3′;
R-5′-aggcattcttcacctgctccac-3′
β-actin:
F-5′-tggacttcgagcaagagatggc-3′;
R-5′-acatctgctggaaggtggacag-3′
GAPDH:
F-5′-aaggtgaaggtcggagtcaacg-3′;
R-5′-catgaggtccaccaccctgttg-3′

### 3D Collagen Regression Assays

ECs or ECs with pericytes were suspended in 3D collagen matrices and incubated at 37° C. in serum-free defined media as described^{9,50,51}. Recombinant factors or THP-1 conditioned media were added at the indicated doses to existing 48 or 72 hr. cultures with EC only or EC-pericyte tube networks, respectively. After 24 or 48 hr. for EC only or EC-pericyte tubes, respectively, cultures were fixed in 3% glutaraldehyde or 3% paraformaldehyde. EC tube areas were traced and quantitated as described. In some cases, neutralizing antibodies were added (10 μg/ml). For signaling experiments, lysates were prepared from control vs. regressing 3D cultures at different time points.

### Imaging, Microscopy, and Data Analysis

Stained cultures were photographed using an inverted microscope and imaging software (Olympus) while time-lapse videomicroscopy of living cells was performed using a DMI6000B microscope (Leica) and MetaMorph software (Molecular Devices). Confocal images were taken using Leica SP8 LIGHTNING White light laser confocal laser scanning microscope using LAS software (Leica). LAS software and Fiji (Image J) were used to reconstruct confocal Z stacks and create rotating movies.

### Statistical Analysis

Statistical data analysis was performed using Microsoft Excel (Microsoft). Variances were obtained and student t tests were used to compare means between conditions while statistical significance was set at a minimum of p<0.05. All analyses were obtained using a minimum of n≥6 fields per experiment, and >3 validating experimental replicates in total. Unpaired, two-tailed t tests were performed for all statistical comparisons of hyaloid vessel numbers and measurements using GraphPad Prism 7.

### Results

### Identification of Key Pro-Regressive Factors for Capillary Tube Networks

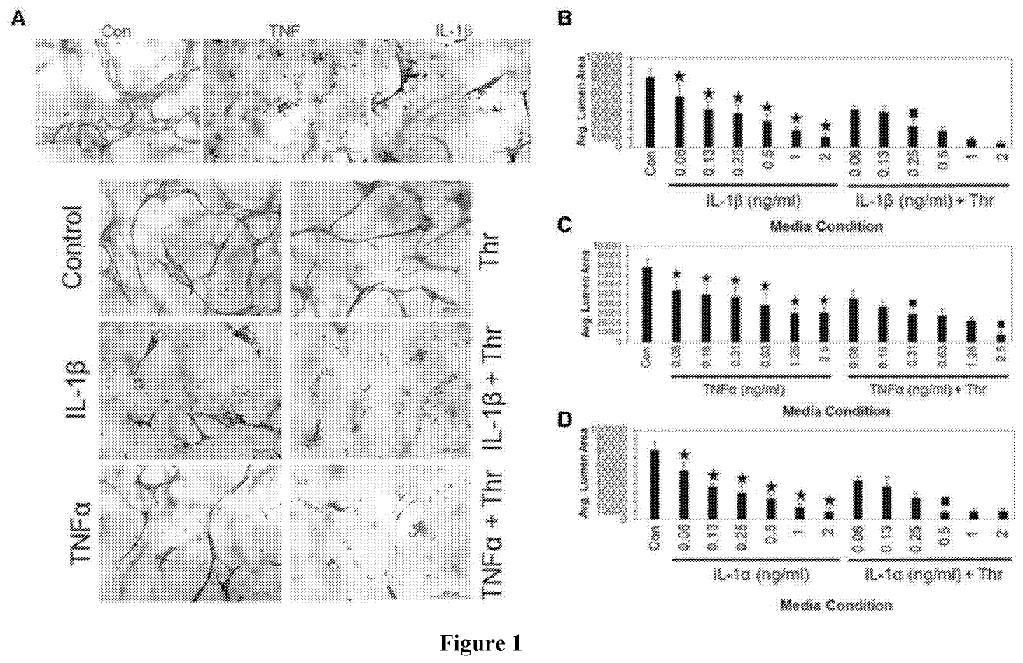

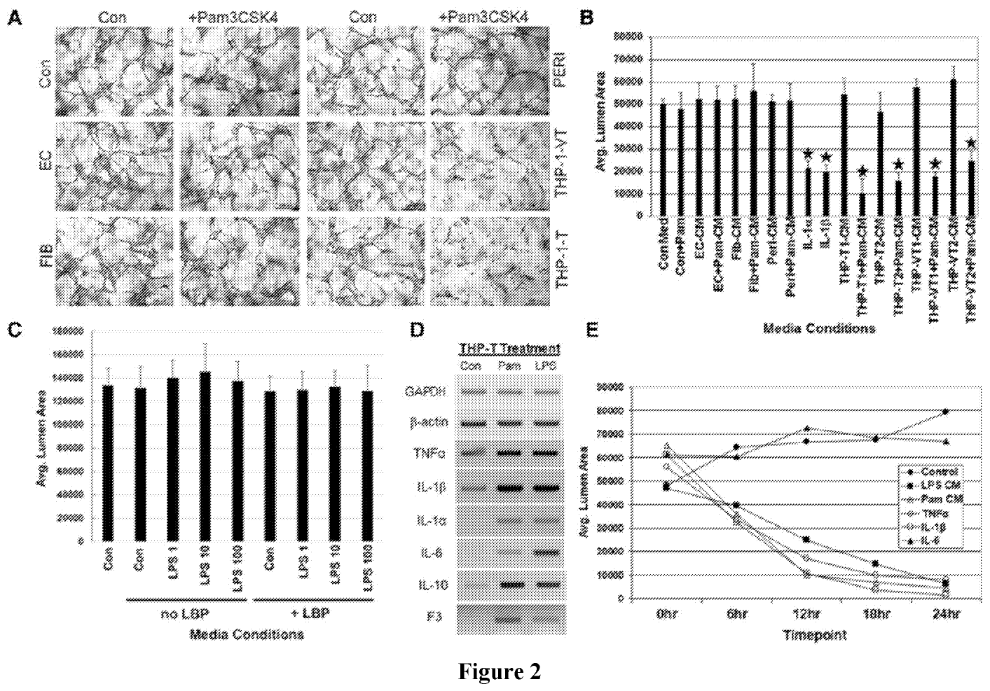

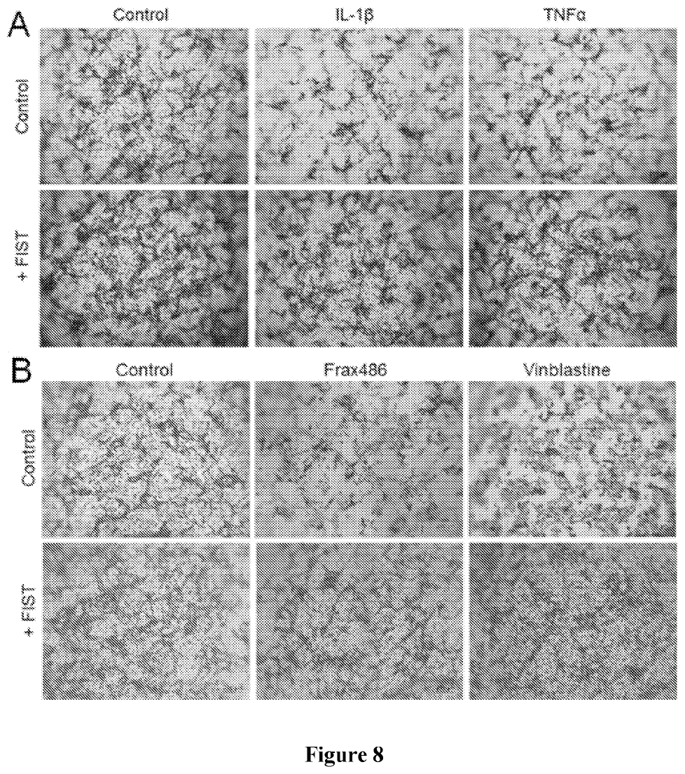

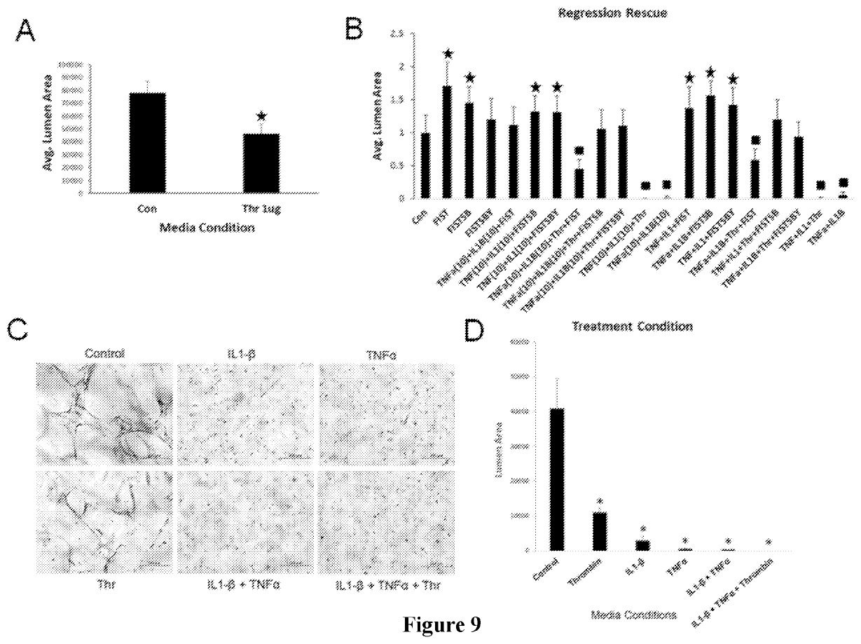

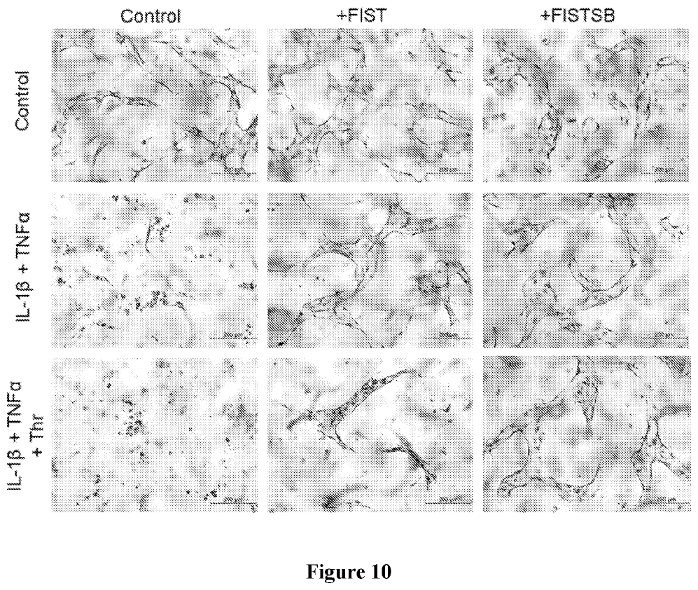

Studies to define the factors and signals that promote endothelial cell (EC) lumen and tube formation, EC sprouting and EC-pericyte tube co-assembly have been performed under serum-free defined conditions so that the functional role of individual molecules that regulate these processes can be assessed. Disclosed herein, the factors that control capillary regression are defined. Capillary regression is a process that has been largely neglected despite the likelihood that it may present a fundamental pathogenic feature of major human diseases where capillary disassembly and regression are known to occur (i.e. hypertension, diabetes, ischemic disease in many tissues, neurodegenerative diseases, and malignant cancer). Screening of many factors to identify stimulators of EC tubulogenesis or sprouting, led to the discovery that IL-1β, IL-la, TNFα and thrombin (individually and their combinations) directly cause capillary tube collapse and regression (FIG. 1, FIG. 8, and FIG. 9). EC tube networks were allowed to form for 48 hr. (vasculogenesis type of assay) and then added these agents in dose-response experiments, showing that they potently induce EC tube regression (at low ng levels) (FIG. 1). Real-time movies were performed to visualize these pro-regressive activities. This experimental approach has utilized assays that mimic vasculogenesis or angiogenic sprouting and these factors induce regression equally well in both assay systems. In the angiogenic sprouting system, TNFα and IL-1β induce degeneration of the invading sprouts, but also the EC monolayer surface where the sprouts originated (FIG. 9A). Addition of thrombin by itself is less effective (FIG. 1, FIG. 10A), but in combination with IL-1β, IL-1α, or TNFα, they potently and rapidly cause EC tube regression (in less than 24 hrs.) (FIG. 1). In contrast, the addition of IL-6, which is co-induced in macrophages along with IL-1 and TNF, does not have pro-regressive activity (FIG. 2E). These studies have been performed primarily in 3D collagen matrices, but the same result is also observed in 3D fibrin matrices. Furthermore, these pro-regressive factors have been added, individually and in combination from the beginning of culture and show that their presence markedly inhibits the formation of EC tube networks with complete loss and death of ECs (FIG. 10C,D). These data indicate that the pro-inflammatory molecules, IL-1, TNFα and thrombin, individually and in combination are potent inducers of EC tube regression in 3D matrices.

### Toll-Like Receptor Activation Leads to Macrophage Production of Pro-Regressive Activities

Since IL-1 and TNF are known to be induced following macrophage exposure to toll-like receptor ligands, such as Pam3CSK4 and lipopolysaccharide (LPS), human macrophages were treated with these ligands to determine if capillary regression promoting activities could be detected. Neither Pam3CSK4 or LPS by themselves (with and without the presence of LPS-binding protein, LBP) had any ability to directly cause EC tube regression (FIG. 2A-C). However, addition of these TLR ligands to macrophages rapidly induced (within 4 hr) highly potent pro-regressive activities in the macrophage conditioned medium, while control macrophage media did not (FIG. 2A, B, FIG. 8B). These media can be used at 1:25-1:100 dilutions and have potent regressive activities suggesting that the factors within this media are highly active. Addition of Pam3CSK4 to other cell types including ECs, pericytes and fibroblasts, failed to induce pro-regressive activities in their conditioned media (FIG. 2A,B). Pam3CSK4- or LPS-induced macrophage media was also assessed to see if it would affect EC tube regression in the presence of pericytes. Rapid loss and regression of EC tubes was observed within the EC-pericyte co-cultures (FIG. 8C), but importantly, pericytes persist and increase in number due to the presence of these pro-regressive activities.

### Identification of the Macrophage Pro-Regressive Activities as IL-1β and TNFα

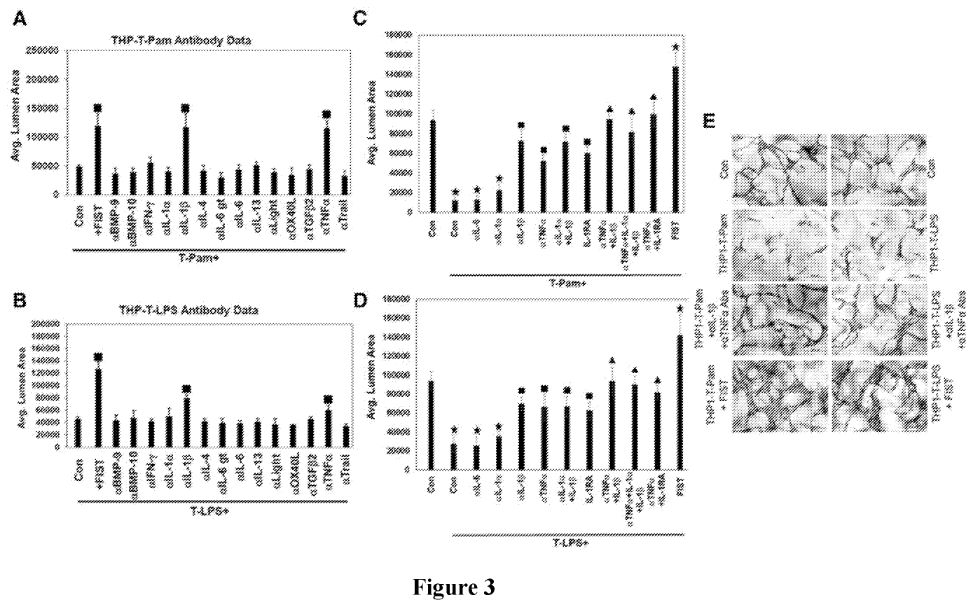

The potent pro-regressive activities within Pam3CSK4- or LPS-induced macrophage conditioned media were identified. Gene expression studies were performed to assess if Pam3CSK4 or LPS affected mRNA expression of inflammatory cytokines and demonstrated marked upregulation of TNFα, IL-1β, IL-la, IL-6 and IL-10 (i.e. an anti-inflammatory cytokine) as well as tissue factor (i.e. F3), which could lead to local thrombin production during a tissue injury response (FIG. 2D). The time course of EC tube regression in response to macrophage conditioned media vs. IL-1β and TNFα was compared and showed that they are directly overlapping (FIG. 2E). In contrast, IL-6 addition had no regressive effect and was just like control cultures. A series of blocking antibodies to pro-regressive factors and various controls were screened to determine if they could neutralize the pro-regressive activity of the macrophage conditioned media. Using either the Pam3CSK4- or LPS-induced media, only the blocking antibodies directed to IL-1β or TNFα were able to neutralize the pro-regressive activity (FIG. 3A, B). Despite the induction of IL-la by Pam3CSK4 and LPS in macrophages (FIG. 2D) and its powerful ability to induce EC tube regression (FIG. 1, FIG. 8A), it did not appear that IL-la was responsible for the macrophage media-derived pro-regressive activity. To further examine the role of IL-1β and TNFα in promoting regression together, their blocking antibodies were added in combination, and show that they completely neutralize the pro-regressive activity of both media compared to controls and anti-IL-6 (FIG. 3C, D). This was also accomplished by adding a combination of IL-1RA, a protein inhibitor of IL-1 signaling, and blocking antibodies to TNFα (FIG. 3C, D).

### Identification of Pharmacologic Inhibitors of EC Tube Regression

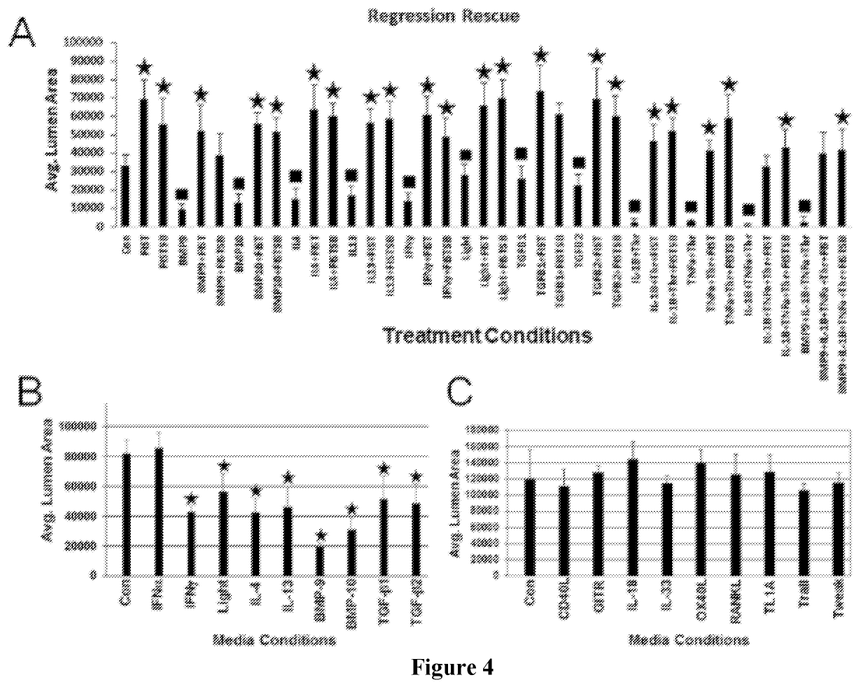

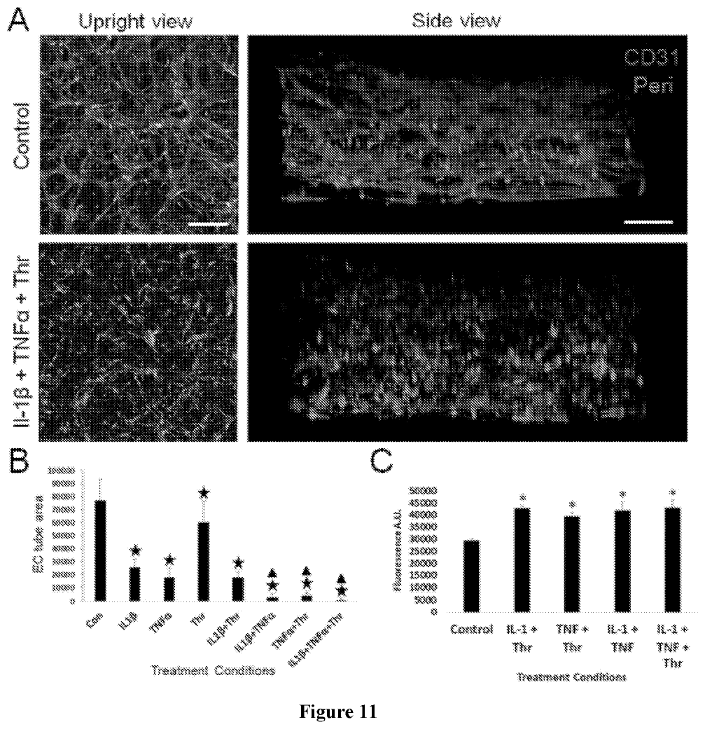

Pharmacologic inhibitors that would antagonize EC tube regression in response to pro-regressive factors were identified. After screening hundreds of combinations of inhibitors or agonists, a four-drug combination (i.e. FIST) was identified that completely rescued the pro-regressive influence of IL-1β, TNFα, and the pro-regressive macrophage media (FIG. 3, FIG. 4, FIG. 8, FIG. 9, FIG. 10). FIST consists of forskolin and IBMX (to stimulate cyclic AMP levels), SB239 (to inhibit p38 Map kinase), and tubacin (to inhibit the tubulin deacetylase, HDAC6). In addition, a fifth drug (SB415286) (to inhibit GSK3(3) was identified, which when added to FIST (i.e. FISTSB), can protect better against the combination of IL-1β and TNFα and especially when thrombin is added with these cytokines (FIG. 4A, FIG. 10A, FIG. 11). These findings were also evaluated and confirmed using real-time movies. This is also true when higher doses of IL-1β and TNFα are added in combination (FIG. 10A), which might mimic severe pathologic conditions resulting from systemic pro-inflammatory macrophage activation along with increased vascular permeability to generate thrombin. Finally, the FIST or FISTSB drug combinations can rescue back to control or above control, the pro-regressive factors or combination of factors identified herein (FIG. 4A). These data may indicate that the pro-regressive factors identified herein appear to stimulate EC tube regression through a common signaling pathway that is antagonized by FIST and FISTSB. These findings also have potential therapeutic implications going forward to protect EC-lined tubes from injurious stimuli during major pathologic events.

Identification of Additional Factors that Stimulate EC Tube Regression

As a part of the screening process to identify other factors that promote EC tube regression, inflammatory cytokines were also screened, including members of the IL-1 and TNF cytokine superfamilies as well as other growth factors known to impact the vasculature (FIG. 4B,C). These were added to 48 hr. cultures of established EC tube networks. This screen was performed at both 40 and 10 ng/ml for each of these factors. FIGS. 4B and C show the data for the 40 ng/ml screen (FIG. 4B,C), however each of the pro-regressive factors identified demonstrated significantly increased regression at both doses and for the factors that did not promote regression, they failed to induce regression at either dose. The pro-regressive factors identified in this screen include IFNγ, IL-4, IL-13, Light, BMP-9, BMP-10, TGFβ1 and TGFβ2, and a long list of factors that did not have any activity, such as IFNα (FIG. 4B). This is a surprisingly large number of pro-regressive factors, but it is an important analysis since individual or combinations of these factors might be present in specific pathological contexts where they might contribute to capillary regression events. Blocking antibodies to each of these factors failed to block Pam3CSK4 and LPS-induced macrophage-derived pro-regressive activities and only antibodies directed to IL-1β and TNFα were able to neutralize these activities (FIG. 3). However, the pharmacologic inhibitor combinations, FIST and FISTSB, both were capable of completely rescuing the regression effects of all the known pro-regressive factors were identified herein (FIG. 4A). Comparatively, IL-1β and TNFα are more potent pro-regressive factors than the other factors described herein.

### IL-1β, TNFα, and Thrombin, and Their Combinations, Selectively Induce Regression of EC Tube Networks Without Loss of Pericytes

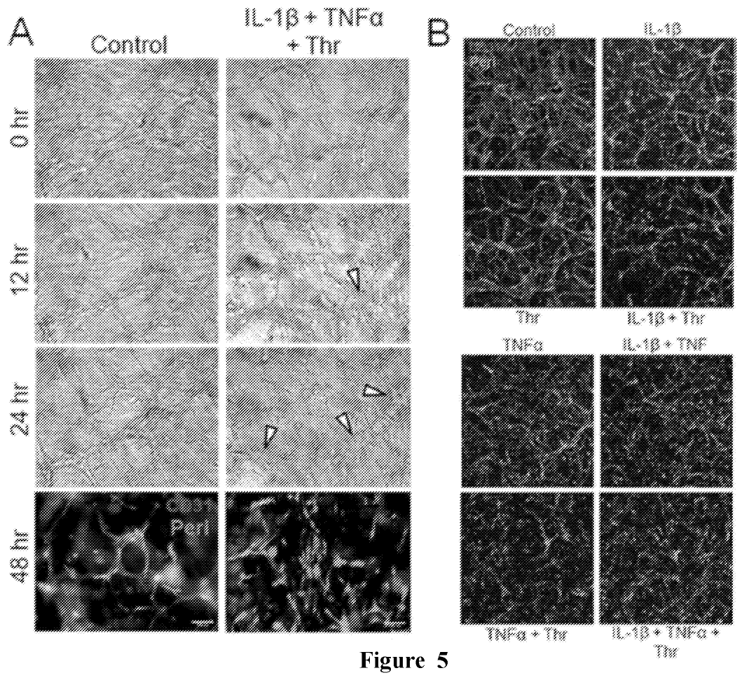

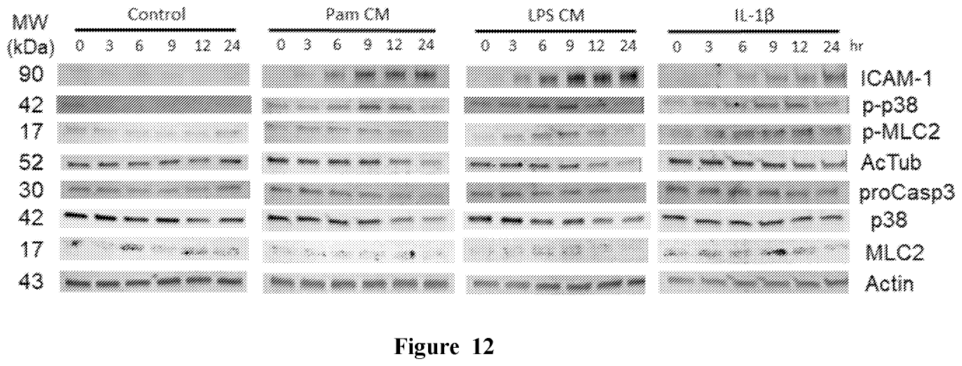

As described herein, macrophage conditioned medium could induce selective loss of EC-lined tubes, while sparing adjacent pericytes (FIG. 8C). These findings were investigated in more detail where defined individual and combinations of IL-1β, TNFα, and thrombin were added to established EC-pericyte co-cultures after 72 hr. of culture (FIG. 5, FIG. 12). Real-time movies were performed which demonstrate the selective regression of EC tubes, while leaving the pericytes viable, migratory and proliferative around the degenerating tubes. The addition of thrombin in combination with IL-1β and thrombin leads to collapse of EC tubes and pericytes remain associated and cluster together with the collapsing tubes and dying ECs (FIG. 5A). Over time, the pericytes migrate out and proliferate in response to the pro-regressive factors. This condition, as well as the controls and other combinations of these three factors were added to EC-pericyte co-cultures at 72 hr., and after 48 hr., cultures were fixed and stained for CD31 to detect the extent of EC tube networks (FIG. 5). EC tube area was quantitated and showed marked tube regression in response to each factor individually and, also in combination (FIG. 12A). In addition, pericyte numbers were quantitated in these cultures and demonstrate an increase in pericyte numbers in response to the pro-regressive factors, despite the marked loss of ECs during tube regression (FIG. 12B).

### Defining a Capillary Regression Signaling Signature in Response to IL-1β, TNFα and Thrombin

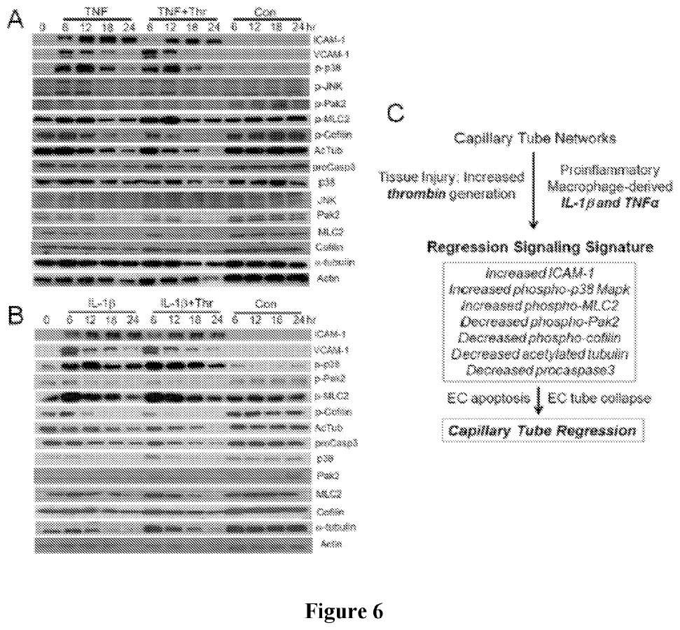

Signaling pathways that underlie the capillary regression process described herein were also evaluated. Lysates from control vs. regressing cultures in response to TNFα, IL-1β, TNFα+thrombin, and IL-1β+thrombin over a 24-hr. time course of regression were prepared and Western blot analyses were performed (FIG. 6). Strong upregulation of ICAM-1 and VCAM-1 was observed, along with increased levels of phospho-p38, phospho-JNK, and phospho-MLC2 during the time course of EC tube regression compared to the control EC tubes. In contrast, decreased levels of phospho-Pak2, phospho-cofilin, acetylated tubulin and procaspase3 was observed in the regressing tubes compared to the control tubes (FIG. 6). In the TNFα+thrombin or the IL-1β+thrombin conditions (which showed increased regression relative to TNFα or IL-1β alone), there appears to be increased phospho-MLC2 and decreased phospho-Pak2, phospho-cofilin, acetylated tubulin and procaspase3 compared to the TNF or IL-1 only conditions. To further investigate this signaling signature, the pro-regressive influence of TNFα and IL-1β using the Pak2 inhibitor, Frax486, or the microtubule depolymerizing drug, vinblastine (which decreases tubulin acetylation) was attempted to be mimicked. Addition of either Frax486 or vinblastine to EC tubes led to marked regression in a manner that strongly resembles that observed following addition of either TNFα or IL-1β (FIG. 9B). Addition of FIST rescued the pro-regressive influence of both drugs and the cytokines (FIG. 9). In further support of these findings, macrophage conditioned media from Pam3CSK4- or LPS-treated cells (and compared to IL-1β alone in the same experiment) caused the same pattern of signals that define the capillary regression signaling signature observed herein. It is important to state that this regression signaling signature is completely distinct (and is the opposite) from that which was previously identified as a signaling signature controlling EC tube formation and maturation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-48. (canceled)

49. A method of treating a disease in a patient in need thereof, the method comprising administering to the patient a therapeutically effective amount of an inhibitor of capillary tube regression.

50. The method of claim 49, further comprising confirming the presence of a capillary tube regression in the patient.

51. The method of claim 49, wherein the disease comprises ischemia, an infarction, hypertension, diabetes, a malignant cancer, a neurodegenerative disease, a wound repair response, atherosclerosis, a pro-inflammatory disease, a pro-thrombotic disease, a viral infection, a bacterial infection, a respiratory disease, or any combination thereof.

52. The method of claim 51, wherein the pro-inflammatory disease comprises arthritis, Crohn's disease, psoriasis, or any combination thereof.

53. The method of claim 51, wherein the viral infection is a SARS-CoV-2 infection, influenza, pneumonia, or any combination thereof.

54. The method of claim 51, wherein the pro-thrombotic disease comprises deep vein thrombosis, pulmonary embolism, and any combination thereof.

55. The method of claim 51, wherein the respiratory disease is coronavirus disease 2019 (COVID-19), acute respiratory distress syndrome (ARDS), or any combination thereof.

56. The method of claim 49, wherein the inhibitor comprises a pharmacologic agent, an antibody, an inhibitor of thrombin generation, or any combination thereof.

57. The method of claim 56, wherein the pharmacologic agent is a small molecule.

58. The method of claim 57, wherein the small molecule comprises forskolin, 3-isobutyl-1-methylxanthine (IBMX), SB239063, tubacin, TCS-HDAC6, tubastatin, SB415286, SB431542, K02288, or any combination thereof.

59. The method of claim 56, wherein the antibody comprises a neutralizing antibody.

60. The method of claim 59, wherein the neutralizing antibody comprises an antibody directed to IL-1β, TNFα, IFN IL-4, IL-13, or any combination thereof.

61. The method of claim 56, wherein the inhibitor of thrombin generation is a Factor Xa inhibitor, a PAR1 inhibitor, or any combination thereof.

62. The method of claim 49, wherein the step of administering to the patient the therapeutically effective amount of the inhibitor of capillary tube regression causes about 50% to about 100% of a reduction of capillary tube regression.

63. The method of claim 49, wherein the inhibitor of capillary tube regression is administered to the patient at a dose ranging from about 1 mg/kg to about 200 mg/kg.

64. A method of treating a disease in a patient in need thereof, the method comprising administering to the patient a therapeutically effective amount of an inhibitor of a lymphatic tube regression.

65. The method of claim 64, further comprising confirming the presence of lymphatic tube regression in the patient.

66. A pharmaceutical composition comprising an inhibitor of capillary regression, wherein the capillary is a blood-carrying capillary, a lymphatic capillary, or any combination thereof.

67. The pharmaceutical composition of claim 66, wherein the inhibitor comprises a pharmacologic agent, an antibody, an inhibitor of thrombin generation, or any combination thereof.

68. The pharmaceutical composition of claim 66, wherein the pharmacologic agent comprises forskolin, IBMX, SB239063, tubacin, TCS-HDAC6, tubastatin, SB415286, SB431542, K02288, or any combination thereof.
